(19) United States
(12) Patent Application Publication
(10) Pub. No.: US 2024/0216656 A1
(43) Pub. Date: Jul. 4, 2024

(54) MEDICAL DEVICE HAVING STEPPED LEAD WIRE

(71) Applicant: ABBOTT CARDIOVASCULAR SYSTEMS INC., Santa Clara, CA (US)

(72) Inventors: Diarmuid J. Wall, Clonmel (IE); Shane O'Neill, Waterford (IE); Rok Kordez, Clonmel (IE); Joseph Keane, New Ross (IE)

(21) Appl. No.: 18/389,659

(22) Filed: Dec. 19, 2023

Related U.S. Application Data
(60) Provisional application No. 63/477,513, filed on Dec. 28, 2022.

(51) Int. Cl. A61M 25/10; A61F 2/958

(52) U.S. Cl. CPC A61M 25/104; A61F 2/958; A61M 2025/09083

(57) ABSTRACT

A medical device includes a catheter shaft extending along a longitudinal axis and having a shaft lumen. A balloon includes a proximal balloon end coupled to the catheter shaft, an interior in fluid communication with the shaft lumen, and a distal balloon end. A lead wire extends from a proximal wire end through the shaft lumen and the interior to a distal wire end. The lead wire includes a stepped portion between a proximal wire surface and a distal wire surface. The distal balloon end is axially fixed to the proximal wire surface. The proximal wire surface is narrower than the distal wire surface. Other embodiments are described and claimed.

This application claims the benefit of priority of U.S. Provisional Patent Application No. 63/477,513, filed on Dec. 28, 2022, titled “MEDICAL DEVICE HAVING STEPPED LEAD WIRE,” which is incorporated herein by reference in its entirety to provide continuity of disclosure.

### FIELD

Various embodiments of the disclosure relate to medical devices, and more specifically to a balloon dilatation medical device in which a balloon is mounted to a lead wire. Other embodiments are also described.

### BACKGROUND

A conventional percutaneous transluminal angioplasty (PTA) is a minimally invasive procedure in which a blocked (or partially blocked) artery, such as a coronary artery is opened in order to improve blood flow to a heart muscle. During a PTA, a guidewire is advanced into the coronary artery until a distal end of the guidewire crosses a lesion. A dilatation catheter, having an inflatable balloon on the distal portion thereof, is advanced into the coronary artery over the previously introduced guidewire until the balloon of the dilatation catheter is properly positioned across the lesion. Once properly positioned, the dilatation balloon is inflated with inflation fluid one or more times to a predetermined size at predetermined pressures so that the arterial wall expands to open up the vascular passageway. After the balloon is deflated, blood resumes through the dilated artery and the dilatation catheter and the guidewire can be removed therefrom.

Critical Limb Ischemia (CLI) is a severe blockage in a patient's lower extremities (e.g., in a leg, ankle, foot), which as a result may reduce (or stop) blood-flow to the extremities. In particular, CLI may be the result of narrowing of arteries (or arteries that are calcified) over time due to a buildup of fatty deposits called plaque. CLI can cause ulcers or gangrene of a foot, which results in severe pain. Patients having CLI are also at elevated risk of requiring foot amputation as a result of these arterial blockages, such as those below the ankle.

### SUMMARY

A conventional balloon catheter used for a PTA may be unable to effectively access and efficiently dilate a lower extremity artery (e.g., the peroneal artery, the dorsalis pedis artery, plantar arteries, etc.) to address CLI. Unlike coronary arteries for which most (if not all) conventional catheters are designed, lower extremity arteries may be more tortuous and thinner. As a result, conventional catheters may not be flexible enough, and may be too large, to be able to effectively navigate through a lower extremity to perform a PTA at a location where a blockage is located. Therefore, there is a need for a dilatation catheter that is capable of navigating through lower extremity arteries to perform a PTA or stenting procedure.

The present disclosure provides a balloon dilatation medical device (or catheter) that can treat stenotic arterial occlusions in lower extremity arteries. Optionally, the device may be a stent delivery system having a stent mounted on the balloon dilatation medical device.

In an embodiment, a medical device includes a catheter shaft extending along a longitudinal axis and having a shaft lumen. The medical device includes a balloon including a proximal balloon end coupled to the catheter shaft. The balloon includes an interior in fluid communication with the shaft lumen. The balloon has a distal balloon end. The medical device includes a lead wire extending from a proximal wire end through the shaft lumen and the interior to a distal wire end. The lead wire includes a stepped portion between a proximal wire surface and a distal wire surface. The distal balloon end is axially fixed to the proximal wire surface. The proximal wire surface is narrower than the distal wire surface.

In one embodiment, a medical device includes a catheter shaft extending along a longitudinal axis. The catheter shaft includes a proximal shaft portion having a proximal shaft end, a distal shaft portion coupled to the proximal shaft portion and having a distal shaft end distal to the proximal shaft portion, and a shaft lumen. The medical device includes a balloon having a proximal balloon end coupled to the distal shaft end such that an interior of the balloon is in fluid communication with the shaft lumen. The balloon has a distal balloon end. The medical device includes a lead wire extending from a proximal wire end through the shaft lumen and the interior to a distal wire end. The distal balloon end is axially fixed to the lead wire. The proximal wire end is located in the shaft lumen within the proximal shaft portion.

In one embodiment, a method of manufacturing a medical device is provided. The method includes grinding a lead wire having a longitudinal axis to form a stepped portion having a proximal wire surface and a distal wire surface. The method includes bonding a distal balloon end of a balloon onto the proximal wire surface such that a balloon outer surface of the balloon has a same radial distance from the longitudinal axis as the distal wire surface.

The above summary does not include an exhaustive list of all embodiments of the disclosure. It is contemplated that the disclosure includes all systems and methods that can be practiced from all suitable combinations of the various embodiments summarized above, as well as those disclosed in the Detailed Description below and particularly pointed out in the claims. Such combinations may have particular advantages not specifically recited in the above summary.

### BRIEF DESCRIPTION OF THE DRAWINGS

The embodiments are illustrated by way of example and not by way of limitation in the figures of the accompanying drawings in which like references indicate similar elements. It should be noted that references to “an” or “one” embodiment of this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1 shows a side view of a balloon dilatation medical device, in accordance with an embodiment.

FIGS. 2A and 2B show cross-sectional side views of the balloon dilatation medical device, in accordance with an embodiment.

FIG. 3 is a cross-sectional view of a balloon dilatation medical device, in accordance with an embodiment.

FIG. 4 shows a cross-sectional side view of a catheter shaft of a balloon dilatation medical device, in accordance with an embodiment.

FIG. 5 is a flowchart of a method of manufacturing a balloon dilatation medical device, in accordance with an embodiment.

### DETAILED DESCRIPTION

Embodiments describe a medical device for treating stenotic arterial occlusions in lower extremity arteries. The medical device may, however, be used in other applications, such as coronary percutaneous transluminal angioplasty (PTA) cases. Thus, reference to the implantable lead as being used for critical limb ischemia (CLI) is not limiting.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal to” may indicate a first direction away from a reference point. Similarly, “proximal to” may indicate a location in a second direction away from the reference point and opposite to the first direction. Such terms are provided to establish relative frames of reference, however, and are not intended to limit the use or orientation of a medical device to a specific configuration described in the various embodiments herein.

In an aspect, a medical device for treating stenotic arterial occlusions in lower extremity arteries can include a catheter shaft, a balloon, and a lead wire. The balloon can be mounted on the catheter shaft and the lead wire. More particularly, the lead wire can extend through at least a portion of the catheter shaft, and entirely through the balloon, to terminate distal to the balloon. The lead wire can have a stepped portion, and the balloon can be axially fixed to the lead wire near the stepped portion. For example, a distal end of the balloon can abut the stepped portion. The stepped portion can therefore provide a smooth transition into an outer surface of the balloon. Furthermore, the lead wire can support the balloon while allowing the balloon to reduce to a smaller diameter than, for example, the catheter shaft. Accordingly, the medical device can include a low profile and have smooth outer surface transitions that allow the medical device to be readily tracked into lower extremity arteries to treat stenotic arterial occlusions.

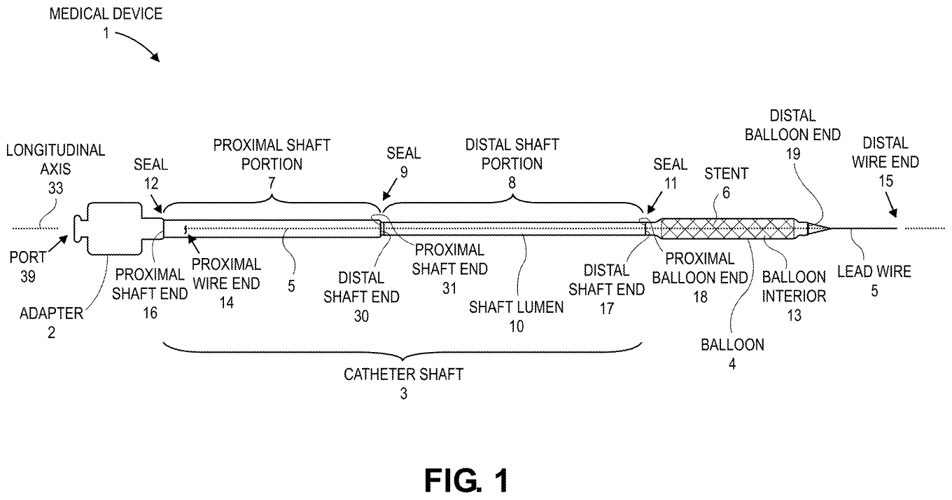

FIG. 1 shows a side view of a medical device 1 (e.g., a balloon dilatation catheter or a stent delivery system), in accordance with an embodiment. The device 1 includes an adapter 2, a catheter shaft 3, a balloon 4, a stent 6 (optionally), and a lead wire 5. In some embodiments, the device may include less or more components. For example, the device may not include the stent 6, in which case the medical device may be used for a PTA (e.g., in a lower extremity artery) in which dilatation is performed at a location with arterial occlusion but without stenting the location. More about the stent is described herein. As shown, the device includes a longitudinal axis 33 extending centrally through (at least some of) the components of the device. In particular, the axis extends through the adapter 2, the catheter shaft 3, the balloon 4, and (at least a portion of) the lead wire 5.

The catheter shaft 3 includes a shaft lumen 10 (e.g., a hollow interior), and extends along the longitudinal axis 33 (e.g., which may be centrally orientated within the shaft lumen, as described herein). In particular, the shaft 3 extends from a proximal shaft end 16 to a distal shaft end 17. The shaft includes a proximal shaft portion 7 and a distal shaft portion 8, where the proximal shaft portion is coupled to the adapter 2 and the distal shaft portion is coupled to the balloon 4. Specifically, a proximal shaft end 16 of the proximal portion 7 is coupled (e.g., to a distal portion of) the adapter 2. A seal 12 can join the proximal shaft portion 7 to the adapter 2. In one embodiment, the shaft lumen 10 may be in fluid communication with an interior of the adapter 2 (while the adapter and the catheter shaft are coupled together). In addition, a distal shaft end 17 of the (e.g., distal shaft portion 8 of the) catheter shaft 3 is coupled to a proximal balloon end 18 of the balloon 4. A seal 11 can join the distal shaft portion 8 to the balloon 4. Both portions of the shaft are also coupled together. In particular, a distal shaft end 30 of the proximal portion 7 may be fixedly coupled (or bonded) to a proximal shaft end 31 of the distal portion 8, at a seal 9, as described herein. More about the shaft portions being coupled together is described herein. The balloon 4 also includes a balloon interior 13 that is in fluid communication with the shaft lumen (e.g., through the seal 11).

In one embodiment, at least some of the seals 9, 11, and/or 12 may be water-tight seals, such that liquid (e.g., flowing through a shaft lumen 10 of the catheter shaft 3) within the medical device 1 does not leak out into the environment. In which case, each of the seals prevents the shaft lumen 10 from being in fluid communication with the environment. In one embodiment, at least one of the seals may be an interference fit, in which end portions of at least two components described herein may be fitted (e.g., pressed) together to form a friction-based connection. For example, the seal 9 may be an interference fit, where the proximal shaft end 31 of the distal shaft portion 8 is inserted into (received by) the distal shaft end 30 of the proximal shaft portion 7. In another embodiment, at least one of the seals may be a seal weld, which is formed by applying heat to a portion of the medical device 1, and then applying an adhesive. In another embodiment, the seal may be formed using one or more adhesives. In another embodiment, components of the device may be (e.g., fixedly) coupled together using any known conventional method to form any type of seal. In one embodiment, at least some of the components of the device may be fixedly coupled such that they are bonded together, thereby creating fixed seals. This may be the case when the seal 9 is an adhesive seal.

In one embodiment, the catheter shaft 3 may comprise at least one material. In particular, the proximal shaft portion 7 may comprise (or be formed from) a first material, while the distal shaft portion 8 may comprise a second material, which is different than the first material. For example, the proximal shaft portion may be a hypotube that is formed from an alloy (e.g., stainless steel) to provide the tube with (at least some) rigidity. The distal shaft portion may be formed from a polymer or copolymer (e.g., a thermoplastic elastomer, such as polyether block amide). In one embodiment, the proximal shaft portion may have a higher rigidity than the distal shaft portion, thereby allowing the distal shaft portion to navigate a tortuous artery. In another embodiment, the proximal shaft portion 7 and the distal shaft portion 8 may be formed from (one or more of) any type of material. In some embodiments, both portions may be formed from the same material (e.g., both including a thermoplastic elastomer).

As shown, the lead wire 5 extends through the balloon 4 and (at least partially) through the catheter shaft 3. In particular, the lead wire 5 extends (e.g., at least partially along the axis 33) from a proximal wire end 14 through the shaft lumen 10 of the catheter shaft 3 and the balloon interior 13 of the balloon to a distal wire end 15 (which is disposed in the environment). In one embodiment, the lead wire 5 may be a solid piece of material (e.g., an alloy such as stainless steel) and/or may comprise one or more separate wires that are braided together to form the lead wire. In one embodiment, the lead wire 5 may have physical characteristics that allow the wire to extend through the medical device 1 and through an artery. For example, the lead wire may be (at least partially) cylindrically shaped, having a diameter that may range from 0.005 inches to 0.038 inches. In one embodiment, the lead wire may have a preferable diameter of 0.014 inches. In another embodiment, the lead wire may have a preferable diameter of 0.018 inches. A wire with a diameter of 0.014 inches or 0.018 inches may be used to perform dilatation upon small vessels. In another embodiment, the diameter of the wire may preferably be 0.035 inches in cases when a dilatation procedure is performed on larger diameter blood vessels.

As described herein, the balloon 4 includes a proximal balloon end 18 that is coupled to the distal shaft end 17 of the catheter shaft 3. The balloon also includes a distal balloon end 19. The distal balloon end 19 may be (e.g., fixedly) coupled to a portion of the lead wire 5 (e.g., a portion closer to the distal wire end 15 than the proximal wire end 14). In which case, the balloon catheter 1 may be a fixed guide wire catheter in which the lead wire may be fixed (e.g., unable to slide along the longitudinal axis 33) with respect to the medical device 1. As shown, the distal end of the lead wire may extend out of the distal balloon end. As described herein, the distal balloon end is axially fixed to a portion of the lead wire 5. More about how the balloon is fixed to the lead wire is described herein. In one embodiment, the proximal wire end 14 may be located within and (fixedly) coupled to (e.g., in interior surface of the shaft lumen 10 of) the catheter shaft 3. In particular, as shown, the proximal wire end 14 is (fixedly) attached to the proximal shaft portion 7. More about how the proximal wire end is coupled to the catheter shaft is described herein.

In one embodiment, the balloon 4 may comprise one or more materials, which allow the balloon to expand (e.g., increasing its volume) in response to increased pressure within the balloon (e.g., due to liquid being flowed into the balloon), and/or retract in response to a decrease in pressure (e.g., due to the liquid being removed from the balloon). For example, the balloon may comprise a thermoplastic elastomer, such as polyether block amide. In some embodiments, the balloon may be arranged to have an expanded size, which may be between 0.5 mm-4 mm, and a length that may be between 10 mm-30 mm. In one embodiment, the length of the balloon may be expandable, such that its length may increase along the longitudinal axis when at least partially inflated, and may decrease when deflated. In one embodiment, the balloon may have a preferable size and length of 1.5 mm and 20 mm, respectively.

As shown, the medical device includes a stent 6 that is loaded onto the balloon 4 by (an inner surface of) the stent being coupled to (a balloon outer surface 24 of) the balloon, such that the stent at least partially surrounds the balloon. In one embodiment, the stent is a (e.g., mesh) tube that is arranged to be positioned into a narrowed artery (during a stenting procedure performed using the medical device 1) in order to provide sufficient blood flow through the artery. In one embodiment, the stent may comprise one or more materials, such as cobalt chromium or stainless steel. As described herein, the stent may be optional. In particular, the medical device 1 may not be equipped with the stent, and in which case, the medical device may be used to perform a PTA in order to open a blocked artery without placing a stent at the blocked location.

As described herein, the medical device 1 may be arranged to perform a PTA or stenting procedure within an artery within a patient's lower extremity. In particular, after making an incision into (e.g., a leg of) a patient, the distal wire end 15 of the lead wire may be inserted into a blocked artery. A user may guide (navigate) the medical device 1 through the artery, until the balloon 4 reaches a blockage site. The user may then (attach the adapter 2 to the catheter shaft 3 if not already attached, and) attach a syringe onto (e.g., a port 39 of the adapter, at a proximal end of) the adapter 2 that includes a liquid (e.g., saline and/or contrast medium). The user may force the liquid into the balloon interior 13 of the balloon 4, through the shaft lumen 10 of the shaft 3, inflating the balloon 4. The balloon may be deflated by removing the liquid. In one embodiment, the balloon may be inflated several times in order to open the blockage site.

In one embodiment, at least a portion of the medical device may have a hydrophilic coating (top layer) that allows the medical device to traverse through an artery with minimal (or no) friction. For example, the (e.g., distal wire end 15 of the) lead wire 5, the balloon 4, and/or at least a portion of the catheter shaft 3 (e.g., the distal shaft portion 8) may be coated with a hydrophilic material.

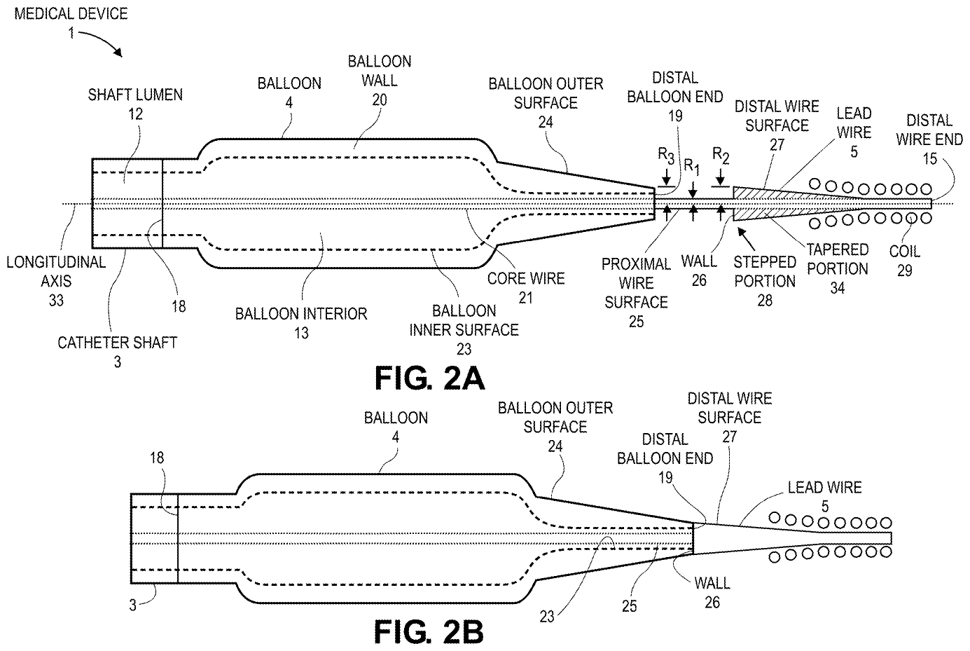

FIGS. 2A and 2B show cross-sectional side views of the balloon dilatation medical device 1, in accordance with an embodiment. Both figures are showing a cross-sectional side view of a portion of the catheter shaft 3, the balloon 4, and the lead wire 5, which is passing through the shaft lumen 12 of the shaft and balloon interior 13 of the balloon. In particular, FIG. 2A is showing the balloon 4 before it is attached to the lead wire 5, and FIG. 2B shows the balloon attached to the wire.

The lead wire 5 may be a stepped lead wire that includes a core wire 21 that is shown as passing through the balloon interior 13 and the shaft lumen 12 (along the axis 33), a stepped portion 28 that includes a wall 26, and a coil 29. The core wire includes a proximal wire surface 25, which may extend proximally from (e.g., the wall 26 of) the stepped portion 28 (e.g., to the proximal wire end 14). The stepped portion 28 is a portion that extends away from the (e.g., core wire 21 having the) proximal wire surface 25, thereby creating a wall 26 which extends tangentially away from the longitudinal axis from the core wire 21). For example, the radius (or radial distance) from the longitudinal axis 33 of the lead wire 5 may differ between the proximal wire surface and the distal wire surface. In particular, the proximal wire surface 25 of the core wire 21 may have a first radius from the longitudinal axis, R₁, which may extend from the stepped portion 28 to the proximal wire end 14. In contrast, the distal wire surface 27 (e.g., at the stepped portion 28) may have a second radius from the longitudinal axis, R₂, which is greater than R₁. Thus, the proximal wire surface 25 is narrower (along a least a portion of the longitudinal axis 33) than the distal wire surface.

In one embodiment, the distal wire surface 27 may taper inward (towards the longitudinal axis 33). For example, the (e.g., stepped portion 28 of the) lead wire 5 includes a tapered portion 34 extending distally from the wall 26 of the stepped portion and towards the distal wire end 15. In particular, the tapered portion 34 tapers from a radial distance of R₂ to a point along the lead wire, between the wall 26 and the distal wire end 15, to radial distance that is equal to or less than R₁. As shown, the coil 29 is disposed on top of the distal wire surface above a portion of the tapered portion 34. In some embodiments, the coil 29 may wrap around a portion of the lead wire, between the stepped portion 28, and the distal wire end 15, as shown. In another embodiment, the tapered portion may end before a proximal end of the coil 29. In another embodiment, the lead wire may not include a tapered portion 28. In which case, the radial distance of the distal wire surface 27 may be R₂ from the wall 26 to the distal wire end 15.

In one embodiment, the core wire 21, the stepped portion 28, and the tapered portion 34 may form the lead wire 5 as a single unit (e.g., formed from a single piece of material, such as stainless steel). In which case, the core wire 21, the stepped portion, and the tapered portion may be structures of the lead wire that comprise the same material. In one embodiment, these elements of the lead wire may be formed through a machine (and/or chemical) process. For example, the lead wire may be mechanically grinded between the distal wire surface 27 and the proximal wire end 14 to reduce the radial size of the lead wire, from R₂ to R₁. More about this grinding process is described herein.

In another embodiment, the core wire 21, which may extend a length of the lead wire, may comprise one material (e.g., stainless steel), while the stepped portion 28 (and the tapered portion 34) may comprise another material (e.g., polyether block amide). In which case, the stepped portion (and/or the tapered portion) may be formed on and entirely surround a portion of the core wire along the longitudinal axis. In which case, the stepped portion may be formed (added) on top of the core wire through one or more mechanical processes.

In addition, the balloon 4 includes a balloon wall 20, which separates the balloon interior 13 from the (ambient) environment. The wall includes a balloon outer surface 24 and a balloon inner surface 23. In one embodiment, the balloon inner surface is arranged to come into contact with the proximal wire surface 25 (when the balloon 4 is bonded to the lead wire 5). In particular, a portion of the balloon inner surface 23 (which is adjacent to the distal balloon end 19) may be attached to the (e.g., proximal wire surface 25 of the) core wire 21. As shown, the distal balloon end 19 has a radial distance from the longitudinal axis of R₃. In one embodiment, at least a portion of the radial distance of the balloon outer surface 24 may be equal to or less than R₃. For example, while the balloon is not inflated, the balloon outer surface may have a radial distance of R₃.

As described herein, FIG. 2A is showing the balloon 4 before it is attached to the lead wire 5. In particular, this figure is illustrating that the (e.g., core wire 21 of the) lead wire is being passed through the (distal balloon end 19) balloon 4 and into the catheter shaft 3 (e.g., during manufacturing of the device 1). FIG. 2B is showing that the balloon 4 is fixed (bonded) to the lead wire. In particular, FIG. 2A is showing the balloon 4 before it is attached to the lead wire 5, and FIG. 2B shows the balloon attached to the wire in which the distal balloon end 19 is axially fixed to the proximal wire surface 25, such that the balloon distal end is directly connected to the proximal wire surface. Specifically, (at least a portion of) the balloon inner surface 23 is fixed to the proximal wire surface 25, while (e.g., a tangential surface of) the distal balloon end 19 abuts the wall 26 of the stepped portion 28. In addition or in lieu of being fixed to the proximal wire surface, the distal balloon end 19 may be fixed to the wall 26. In some embodiments, the balloon may be fixed to the lead wire via an adhesive.

In one embodiment, at least a portion of the balloon outer surface 24 may have a same (or less than) radial distance from the longitudinal axis, as the distal wire surface 27 of the lead wire 5. For instance, R₃ may be equal to R₂, such that while the balloon is fixed to the lead wire the balloon outer surface 24 (e.g., at the distal balloon end 19) is flush with the distal wire surface 27 (at the stepped portion 28). In one embodiment, the outer surface of the balloon may be flush to the distal surface of the wire (e.g., both surfaces having the same radial distance while the distal balloon end 19 and the stepped portion 28 are in direct contact) such that no objects may enter a space between the balloon and the stepped portion. This may prevent objects (e.g., tissue) from being snagged by the medical device, while it is traversing through an artery.

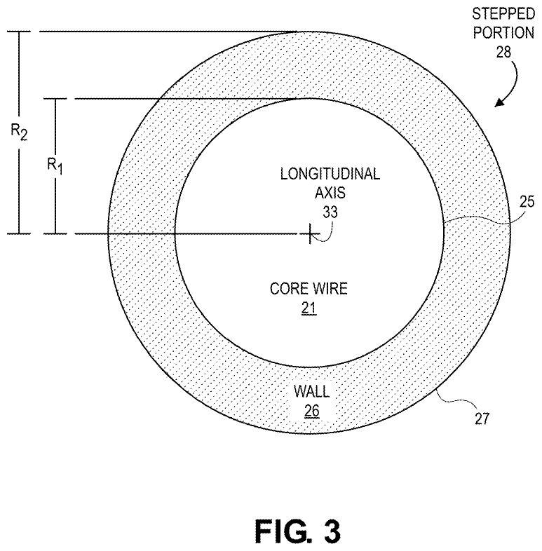

FIG. 3 is a cross-sectional view of a balloon dilatation medical device, in accordance with an embodiment. In particular, this figure is showing a cross-sectional view of the medical device 1 at (or near) the stepped portion 28. It shows that the stepped portion entirely surrounds the core wire 21 along the longitudinal axis 33. It also shows the thickness (radial distance of the core wire) from the longitudinal axis 33 to the surface of the wire (e.g., proximal wire surface 25), as R₁. It also shows the radial distance of the wall 26 of the stepped portion from the axis 33 to the surface of the stepped portion (e.g., distal wire surface 27).

As shown by the cross-section, the core wire 21 and the stepped portion 28 may both have circular cross-sections. In another embodiment, either of the elements may be shaped differently. For example, the core wire 21 may have a rectangular cross-section, while the stepped portion 28 may be a circular cross-section. In some embodiments, the lead wire may have one or more shapes.

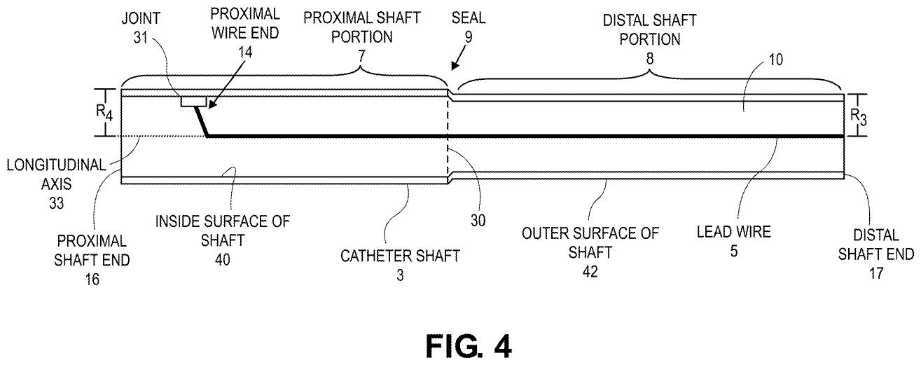

FIG. 4 shows a cross-sectional side view of the catheter shaft 3 of a balloon dilatation medical device 1, in accordance with an embodiment. In particular, this figure is showing a cross-sectional view of the shaft 3 from the proximal shaft end 16 to the distal shaft end 17. The cross-section shows the (e.g., core wire 21 of the) lead wire 5 extending through the distal shaft portion 8 and at least partially through the proximal shaft portion 7 of the catheter shaft 3. Also shown, the proximal wire end 14 of the lead wire is coupled to the inside surface 40 of the shaft 3. Specifically, the proximal end 14 is coupled to the inside shaft via a joint 31. In one embodiment, the joint 31 may be a spot weld, which welds (fixedly couples) the proximal end to the proximal portion 7. In another embodiment, the joint 31 may be an adhesive that couples the proximal end 14 to the inside surface 40 of the shaft 3. As shown, the joint 31 is disposed closer (e.g., within a threshold) to the proximal shaft end 16 than to the distal shaft end 30 at the seal 9. In another embodiment, the joint may be disposed closer to the seal. In some embodiments, the joint may be disposed along the distal shaft portion 8. In another embodiment, the joint may be disposed anywhere within the catheter shaft 3 that is proximal to the balloon 4. In some embodiments, the proximal wire end 14 may not be coupled to the inside surface 40 of the shaft 3. In which case, the proximal wire end 14 may be floating within the catheter shaft 3, allowing the proximal wire end 14 to move within the shaft (e.g., as the medical device traverses through an artery).

As described thus far, the joint 31 may be disposed anywhere within the shaft lumen 10. In another embodiment, the proximal wire end 14 of the lead wire 5 may be disposed at any location within the shaft lumen 10. This may be the case when the proximal end 14 is floating within the shaft by not being fixed to the inner surface 40 of the shaft 3 via a joint. In another embodiment, the lead wire may be fixed to the inside surface of the shaft via a joint that is fixed to a portion of the lead wire other than the proximal wire end 14. For example, a portion of the core wire 21 may be fixed to the inside surface 40 of the shaft 3 within the distal shaft portion 8, while the proximal wire end 14 is disposed within the proximal shaft portion 7.

In some embodiments, the proximal shaft portion 7 may have one or more dimensions that are different than the distal shaft portion 8. For instance, the distal shaft portion 8 may have a first radial distance, R₃, from the longitudinal axis 33 to the outer surface 42 of the shaft 3 (on the distal shaft portion 8), and the proximal shaft portion 7 may have a second radial distance, R₄, from the longitudinal axis 33 to the outer surface 42 of the shaft 3 (on the proximal shaft portion 7). In one embodiment, R₄ may be greater than R₃. In another embodiment, R₃ and R₄ may be the same. In another embodiment, the shaft portions may have a same length (along the longitudinal axis), or may be different. For example, the distal shaft portion 8 may be longer than the proximal shaft portion 7.

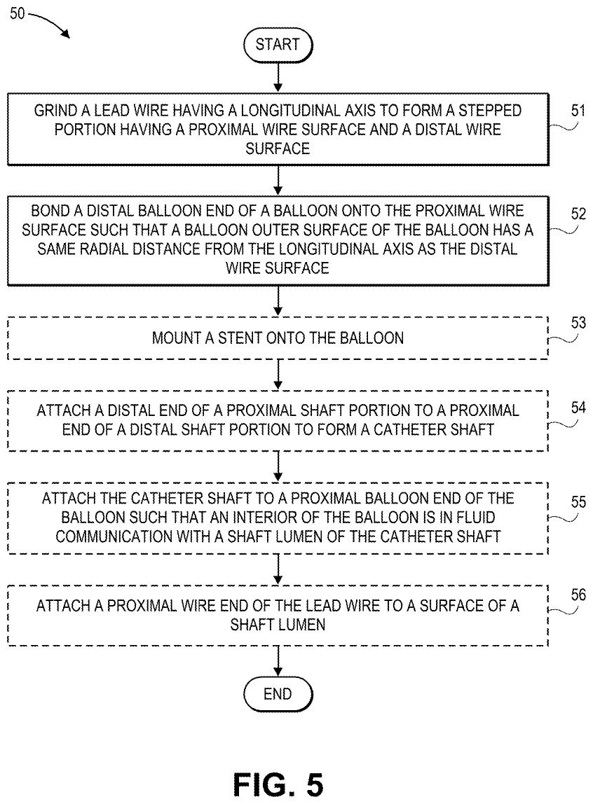

FIG. 5 is a flowchart of one embodiment of a process 50 for manufacturing a balloon dilatation medical device 1. In one embodiment, the process 50 may be performed by a single (e.g., manufacturing) machine or multiple machines to manufacture one or more medical devices 1, illustrated in FIG. 1. In some embodiments, at least some of the operations within this process may be automatically performed by one or more machines. For example, at least some of the operations may be performed without user intervention. In another embodiment, at least some of the operations may be manually performed by one or more users operating one or more machines.

The process 50 begins by grinding a lead wire having a longitudinal axis to form a stepped portion having a proximal wire surface and a distal wire surface (at block 51). As described herein, the lead wire may be a cylindrically shaped piece of material (e.g., stainless steel), which is cut to a particular length. In which case, the lead wire may be grinded such that a radial portion of the lead wire is removed (e.g., through a centerless grinding process). In another embodiment, the radial portion may be removed by pulling the proximal shaft end 16 through one or more dies. In some embodiments, the grinding of the lead wire may result in a portion of the lead wire having a radial distance (e.g., R₁) that is less than another (e.g., stepped) portion (e.g., R₂).

In one embodiment, the pre-grinded lead wire may be a solid cylinder of at least one material, such as stainless steel, having a radial distance from a longitudinal axis that runs through a center of the cylinder of R₂. In which case, the lead wire may be grinded such that the solid cylinder may change (e.g., to varying degrees) about one or more portions of the lead wire to produce the desired structure of the lead wire. For example, referring to FIG. 2A, the grinding may form the stepped portion 28 of the lead wire by removing a radial portion of the lead wire from R₂ to R₁ proximal to the desired location of the stepped portion. Thus, the lead wire may be ground around the circumference of the wire and along a length of a wire from the proximal wire end 14 to a location along the wire at which the stepped portion 28 is desired.

The process 50 bonds a distal balloon end (e.g., 18) onto a proximal wire surface (e.g., 25) such that a balloon outer surface (e.g., 24) of the balloon has a same radial distance from the longitudinal axis as the distal wire surface (at block 52). For example, the balloon is bonded to the lead wire by attaching (e.g., via an adhesive) the balloon inner surface 23 to the core wire 21 and/or attaching the distal balloon end 19 to the wall 26 of the stepped portion 28.

Optionally, the process 50 mounts a stent onto the balloon 4 (at block 53). For instance, the stent 5 may be passed over a portion of the medical device 1 (e.g., having the stent passed over the catheter shaft from the proximal wire end towards the balloon in a distal direction). Optionally, the process 50 attaches a distal end (e.g., 30) of a proximal shaft portion 7 to a proximal end (e.g., 31) of a distal shaft portion (e.g., 8) to form a catheter shaft (e.g., 3) (at block 54). As described herein, the distal shaft portion may be inserted into the proximal shaft portion, forming an interference fit as a seal 9. Optionally, the process 50 attaches the catheter shaft 3 to a proximal balloon end 18 of the balloon 4 such that an interior of the balloon is in fluid communication with a shaft lumen of the catheter shaft (at block 55). Optionally, the process attaches a proximal wire end of the lead wire to a surface of the shaft lumen (at block 56). In particular, referring to FIG. 4, the proximal wire end 14 may be bonded to the inside surface 30 of the shaft 3 via the joint 31. In one embodiment, the joint 31 may be an adhesive or a thermal bond.

Some embodiments may perform variations to at least one of the process 50 described herein. For example, the specific operations of a process may not be performed in the exact order shown and described. The specific operations may not be performed in one continuous series of operations and different specific operations may be performed in different embodiments. For example, the operations within dashed boxes may be optional operations that may not be performed while at least a portion of the process 50 is performed. As an example, the process may omit the operations performed in block 53 to mount (or load) the stent onto the balloon, when a stent is not necessary for use during a procedure for which the medical device is to be used.

As described thus far, the lead wire 5 may be formed by grinding a single piece of material (a stainless steel wire) to form the structure of the wire 5 (e.g., as shown in FIGS. 2A and 2B. In another embodiment, at least some of the structures of the lead wire may be formed separately, using varied materials. For instance, the stepped portion 28 (and/or the tapered portion 34) may be added onto the core wire 21 to form the lead wire 5. In which case, the added structures may be a different material than the core wire (e.g., the portions being polyether block amide, while the core wire being stainless steel. In one embodiment, a molding process may be performed such that the stepped portion and/or the tapered portion are added to the structure. In another embodiment, the structures may be fixedly coupled to the core wire (e.g., through the use of an adhesive).

In another embodiment, the stepped portion 28 and/or the tapered portion 34 may be formed by other methods. As one example, the lead wire 5 may include at least one material layer, as a top layer (e.g., polytetrafluoroethylene (PTFE) that entirely surrounds at least a portion (e.g., of the outer surface) of the core wire 21 along (e.g., a length of the wire along) the longitudinal axis. In one embodiment, the PTFE may provide durability to the lead wire. Thus, the grinding of the lead wire may include removing (stripping) the material top layer off of the core wire, such that a remaining portion of the top layer that is attached to the core wire 21 (which comprises the material layer) is the stepped portion.

While certain embodiments have been described and shown in the accompanying drawings, it is to be understood that such embodiments are merely illustrative of and not restrictive on the broad invention, and the invention is not limited to the specific constructions and arrangements shown and described, since various other modifications may occur to those of ordinary skill in the art. The description is thus to be regarded as illustrative instead of limiting.

To aid the Patent Office and any readers of any patent issued on this application in interpreting the claims appended hereto, the appended claims or claim elements are not intended to invoke 35 U.S.C. 112(f) unless the words “means for” or “step for” are explicitly used in the particular claim.

What is claimed is:

1. A medical device, comprising:
a catheter shaft extending along a longitudinal axis and having a shaft lumen;
a balloon including a proximal balloon end coupled to the catheter shaft, an interior in fluid communication with the shaft lumen, and a distal balloon end; and
a lead wire extending from a proximal wire end through the shaft lumen and the interior to a distal wire end, wherein the lead wire includes a stepped portion between a proximal wire surface and a distal wire surface, wherein the distal balloon end is axially fixed to the proximal wire surface, and wherein the proximal wire surface is narrower than the distal wire surface.

2. The medical device of claim 1, wherein the proximal wire surface has a first radial distance from the longitudinal axis and the distal wire surface has a second radial distance from the longitudinal axis that is greater than the first radial distance.

3. The medical device of claim 2, wherein a balloon outer surface of the balloon at the distal balloon end has a radial distance from the longitudinal axis that is the same or less than the second radial distance.

4. The medical device of claim 1, wherein the distal balloon end is directly connected to the proximal wire surface.

5. The medical device of claim 1, wherein the distal balloon end abuts a wall of the stepped portion.

6. The medical device of claim 5, wherein a balloon outer surface at the distal balloon end of the balloon is flush with the distal wire surface.

7. The medical device of claim 1 further comprising a stent mounted on a balloon outer surface of the balloon.

8. The medical device of claim 1, wherein the catheter shaft includes a distal shaft portion coupled to the proximal balloon end, and a hypotube proximal to the distal shaft portion, and wherein the proximal wire end is located within the hypotube.

9. The medical device of claim 8, wherein the proximal wire end is attached to the hypotube.

10. The medical device of claim 1, wherein the lead wire comprises a core wire that extends a length of the lead wire and is composed of a first material, and wherein the stepped portion is formed on and entirely surrounds a portion of the core wire along the longitudinal axis and is composed of a second material that is different than the first material.

11. A medical device, comprising:
a catheter shaft extending along a longitudinal axis and including a proximal shaft portion having a proximal shaft end, a distal shaft portion coupled to the proximal shaft portion and having a distal shaft end distal to the proximal shaft portion, and a shaft lumen;
a balloon having a proximal balloon end coupled to the distal shaft end such that an interior of the balloon is in fluid communication with the shaft lumen, and a distal balloon end; and
a lead wire extending from a proximal wire end through the shaft lumen and the interior to a distal wire end, and wherein the distal balloon end is axially fixed to the lead wire, and wherein the proximal wire end is located in the shaft lumen within the proximal shaft portion.

12. The medical device of claim 11, wherein the proximal shaft portion is fixedly attached to the distal shaft portion, wherein the proximal shaft portion is formed from a first material and the distal shaft portion is formed from a second material that is different than the first material.

13. The medical device of claim 12, wherein the proximal shaft portion has a first radial distance from the longitudinal axis and the distal shaft portion has a second radial distance from the longitudinal axis, and wherein the second radial distance is different than the first radial distance.

14. The medical device of claim 11, wherein the proximal wire end is fixedly attached to the catheter shaft.

15. The medical device of claim 11, wherein the lead wire comprises a stepped portion between a proximal wire surface and a distal wire surface, and wherein the distal balloon end of the balloon is axially fixed to the proximal wire surface.

16. The medical device of claim 15, wherein the lead wire comprises a stepped portion wherein an outer surface of the distal balloon end has a same radial distance from the longitudinal axis as the distal wire surface.

17. A method, comprising:
grinding a lead wire having a longitudinal axis to form a stepped portion having a proximal wire surface and a distal wire surface; and
bonding a distal balloon end of a balloon onto the proximal wire surface such that a balloon outer surface of the balloon has a same radial distance from the longitudinal axis as the distal wire surface.

18. The method of claim 17 further comprising mounting a stent on the balloon.

19. The method of claim 17 further comprising attaching a catheter shaft to a proximal balloon end of the balloon such that an interior of the balloon is in fluid communication with a shaft lumen of the catheter shaft.

20. The method of claim 19, wherein a distal end of the lead wire extends out of the distal balloon end, and wherein the method further comprises attaching a proximal end of the lead wire to a surface of the shaft lumen.

21. The method of claim 19, wherein the catheter shaft includes a proximal shaft portion of a first material and a distal shaft portion of a second material, and further comprising attaching a distal end of the proximal shaft portion to a proximal end of the distal shaft portion.

22. The method of claim 21, wherein an outer surface of the proximal shaft portion has a first radial distance from the longitudinal axis and an outer surface of the distal shaft portion has a second radial distance from the longitudinal axis, and wherein the second radial distance is different than the first radial distance.

23. The method of claim 17, wherein the wire comprises a core wire composed of a material having a first radial distance from a longitudinal axis, wherein grinding the lead wire comprises reducing a portion of the core wire proximal to the stepped portion to a second radial distance from the longitudinal axis, and wherein the second radial distance is less than the first radial distance.

24. The method of claim 17, wherein the wire comprises a core wire composed of a first material and a top layer that is formed on and entirely surrounds a portion of the core wire along a longitudinal axis, wherein the top layer is composed of a second material, and wherein grinding the lead wire comprises removing a portion of the top layer such that a remaining portion of the top layer is the stepped portion.
